(19) United States
(12) Patent Application Publication
(10) Pub. No.: US 2026/0199664 A1
(43) Pub. Date: Jul. 16, 2026

(54) ELECTRICAL STIMULATION DEVICE

(71) Applicant: UGYM TECHNOLOGY CORP., Hsinchu County (TW)

(72) Inventors: Fu-Hao YU, Hsinchu County (TW); Chin-Chuan HSU, Hsinchu County (TW); Chiu-Hung HSU, Hsinchu County (TW)

(21) Appl. No.: 19/193,586

(22) Filed: Apr. 29, 2025

(30) Foreign Application Priority Data
Jan. 16, 2025 (TW) 114200656

(51) Int. Cl. A61N 1/04; A61N 1/36

(52) U.S. Cl. CPC A61N 1/0456; A61N 1/0492; A61N 1/36021; A61N 1/36034

(57) ABSTRACT

An electrical stimulation device includes at least two electrode patches and a device body. The device body is electrically connected to the electrode patches. The device body includes a signal generation unit and a modulation unit electrically connected to the signal generation unit. The signal generation unit outputs a low-frequency signal and a medium-frequency signal to the modulation unit. The modulation unit adds the low-frequency signal into the medium-frequency signal in a frequency modulation manner so as to generate an interference wave signal, and individually transmits the interference wave signal to the electrode patches.

### CROSS REFERENCE TO RELATED APPLICATIONS

This Non-provisional application claims priority under 35 U.S.C. § 119(a) on Patent Application No(s). 114200656 filed in Taiwan, Republic of China on Jan. 16, 2025, the entire contents of which are hereby incorporated by reference.

### BACKGROUND

### Technology Field

The present disclosure relates to an electrical stimulation device and, in particular, to a medium-frequency electrical stimulation device.

### Description of Related Art

Interference Current therapy (IFC therapy) is a physical therapy method that uses two groups of currents with different frequencies to interfere with each other to generate low-frequency currents. It is achieved by two groups of medium-frequency currents interfering with each other at the treatment site to generate a low-frequency beat frequency (difference frequency). This beat frequency can penetrate deep into the tissues to achieve the following effects. 1. Blocking pain signals: the low-frequency current can interfere with the transmission of pain signals so as to reduce pain. 2. Promoting the release of endorphins: to stimulate the nervous system to release endorphins, thereby producing a natural analgesic effect. 3. Increasing blood circulation: to dilate blood vessels so as to increase local blood flow, thereby helping supply nutrients and excrete metabolic wastes. 4. Reducing muscle spasms: to relieve muscle tension so as to reduce spasms and stiffness.

The conventional electrical stimulator requires four patches to be placed in two pairs opposite to each other and crossly attached to the human body in order to achieve the effect of interference wave. For example, as shown in FIG. 1A and FIG. 1B, wherein FIG. 1A is a schematic diagram showing a conventional electrical stimulator having two pairs of electrode patches 21a to 21d attached to a human body 3, and FIG. 1B is a schematic diagram showing the waveform of interference wave generated by the two pairs of electrode patches 21a to 21d of FIG. 1A in a crossing arrangement. As shown in FIG. 1A, the electrode patches 21a and 21b form a first pair of electrode patches, and the electrode patches 21c and 21d form a second pair of electrode patches. These two pairs of electrode patches 21a to 21d are attached to the human body 3 in a crossing arrangement and are simultaneously applied with different medium frequencies. Due to the frequency difference, a low-frequency interference wave 2 as shown in FIG. 1B is generated. The interference wave 2 is mainly generated at the intersection between the two pairs of electrode patches 21a to 21d, and the effect of the interference wave 2 decreases as it goes outwardly away from the center of the intersection.

### SUMMARY

An objective of this disclosure is to provide an electrical stimulation device, which can use only two electrode patches to generate more uniform interference wave than conventional electrical stimulators, and generate the interference wave with larger coverage area.

To achieve the above, an electrical stimulation device of this disclosure includes at least two electrode patches and a device body electrically connected to the electrode patches. The device body includes a signal generation unit and a modulation unit electrically connected to the signal generation unit. The signal generation unit outputs a low-frequency signal and a medium-frequency signal to the modulation unit. The modulation unit adds the low-frequency signal into the medium-frequency signal in a frequency modulation manner so as to generate an interference wave signal, and individually transmits the interference wave signal to the electrode patches.

In one embodiment, the electrical stimulation device further includes at least two connection wires arranged corresponding to the at least two electrode patches, and the device body is electrically connected to the electrode patches through the at least two connection wires, respectively.

In one embodiment, the electrical stimulation device further includes a power supply unit electrically connected to the signal generation unit and the modulation unit, and the power supply unit provides an electric power for operating the electrical stimulation device.

In one embodiment, the electrical stimulation device further includes a control unit electrically connected to the signal generation unit, the modulation unit and the power supply unit, and the control unit is configured to control an operation of the electrical stimulation device.

In one embodiment, the low-frequency signal has a frequency between 1 Hz and 250 Hz.

In one embodiment, the medium-frequency signal has a frequency between 1 k Hz and 10 k Hz.

In one embodiment, the device body is configured with a mode switch button for switching an operation mode of the electrical stimulation device.

In one embodiment, the device body is configured with an intensity adjustment button for adjusting a peak-to-peak voltage value of the interference wave signal of the electrical stimulation device.

In one embodiment, the electrical stimulation device includes four of the electrode patches.

In one embodiment, the electrical stimulation device is a transcutaneous electrical nerve stimulator (TENS).

As mentioned above, in the electrical stimulation device of this disclosure, the device body is electrically connected to at least two electrode patches and includes a signal generation unit and a modulation unit electrically connected to the signal generation unit. The signal generation unit outputs a low-frequency signal and a medium-frequency signal to the modulation unit, and the modulation unit adds the low-frequency signal into the medium-frequency signal in a frequency modulation manner so as to generate an interference wave signal, and individually transmits the interference wave signal to the electrode patches. Compared with the conventional electrical stimulator, the electrical stimulation device of this disclosure can use only two electrode patches to generate more uniform interference wave than the conventional electrical stimulators, and can generate the interference wave with larger coverage area.

### BRIEF DESCRIPTION OF THE DRAWINGS

The disclosure will become more fully understood from the detailed description and accompanying drawings, which are given for illustration only, and thus are not limitative of the present disclosure, and wherein:

FIG. 1A is a schematic diagram showing a conventional electrical stimulator having two pairs of electrode patches attached to a human body;

FIG. 1B is a schematic diagram showing the waveform of interference wave generated by the two pairs of electrode patches of FIG. 1A in a crossing arrangement;

FIG. 2A is a schematic diagram showing an electrical stimulation device according to an embodiment of this disclosure;

FIG. 2B is a block diagram of the electrical stimulation device of FIG. 2A;

FIG. 2C is a schematic diagram showing the waveform of interference wave generated by the electrical stimulation device according to the embodiment of this disclosure; and

FIGS. 3 to 5 are schematic diagrams showing different coverage areas of the interference waves generated by the electrical stimulation device according to the embodiment of this disclosure.

### DETAILED DESCRIPTION OF THE DISCLOSURE

The present disclosure will be apparent from the following detailed description, which proceeds with reference to the accompanying drawings, wherein the same references relate to the same elements.

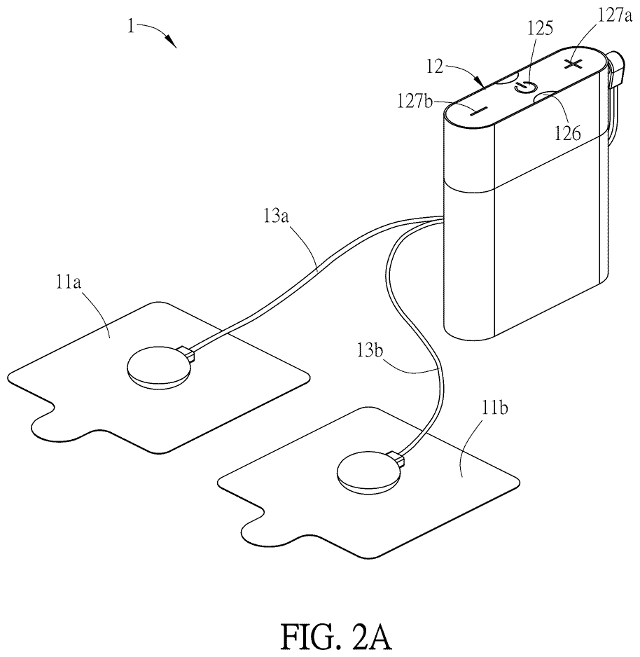

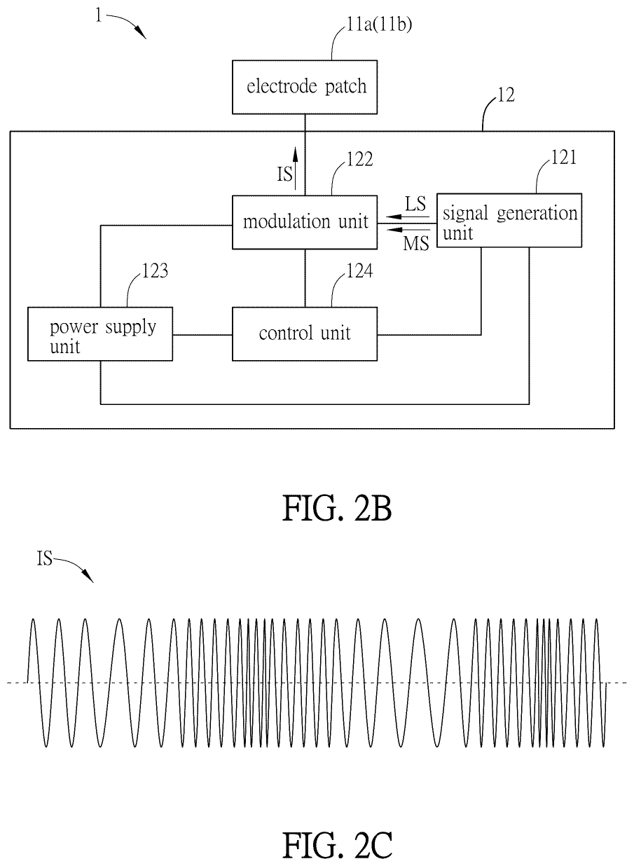

FIG. 2A is a schematic diagram showing an electrical stimulation device 1 according to an embodiment of this disclosure, FIG. 2B is a block diagram of the electrical stimulation device 1, and FIG. 2C is a schematic diagram showing the waveform of interference wave generated by the electrical stimulation device 1. FIGS. 3 to 5 are schematic diagrams showing different coverage areas of the interference waves generated by the electrical stimulation device 1. To be noted, FIG. 2C is only for illustrating the waveform of interference wave generated by modulating the medium-frequency signal MS with the low-frequency signal LS as shown in FIG. 2B, and is does not represent the actual waveform of the interference wave actually used. In addition, the interference wave shown in FIG. 2C is a sine wave as an example, but this disclosure is not limited thereto. In other embodiments, the interference wave may be a square wave.

Referring to FIGS. 2A to 2C, the electrical stimulation device 1 of this disclosure is a medium-frequency electrical stimulator, which can also be called a transcutaneous electrical nerve stimulator (TENS). The working principle thereof is to use the medium-frequency current with different frequencies and appropriate intensity changes to perform electrical stimulation through the skin surface to relieve pain symptoms, especially for the pain at deep muscles. The electrical stimulation device 1 of FIG. 2A is, for example, a pocket-sized device. In different embodiments, the electrical stimulation device 1 may be an electrical stimulator placed on a table or standing on the ground, and this disclosure is not limited thereto.

The electrical stimulation device 1 includes at least two electrode patches 11a and 11b, and a device body 12. In addition, the electrical stimulation device 1 may further include at least two connection wires 13a and 13b. For example, FIG. 2A shows that the electrical stimulation device 1 includes two electrode patches 11a and 11b, and two connection wires 13a and 13b, which are arranged corresponding to the two electrode patches 11a and 11b, respectively.

The electrode patches 11a and 11b can be button-type conductive electrode patches with any shape, such as, for example but not limited to, square, rectangular, oval or circular, or any of other shapes.

The device body 12 is electrically connected to the electrode patches 11a and 11b. In this embodiment, the device body 12 is electrically connected to the electrode patch 11a through the connection wire 13a, and electrically connected to the electrode patch 11b through the connection wire 13b. Thus, the device body 12 can transmit the interference wave to the electrode patches 11a and 11b through the connection wires 13a and 13b, respectively. Unlike the conventional electrical stimulator, the device body 12 of the electrical stimulation device 1 of this embodiment can directly transmit the interference wave to the electrode patches 11a and 11b, thereby achieving the effects of relieving muscle tension and reducing muscle spasms and stiffness.

As shown in FIG. 2B, the device body 12 includes a signal generation unit 121 and a modulation unit 122. In this embodiment, the device body 12 may further include a power supply unit 123 and a control unit 124. In this case, the signal generation unit 121, the modulation unit 122, the power supply unit 123 and the control unit 124 are all configured inside the device body 12.

The signal generation unit 121 is electrically connected to the modulation unit 122. The signal generation unit 121 can output a low-frequency signal LS and a medium-frequency signal MS to the modulation unit 122. After receiving the low-frequency signal LS and the medium-frequency signal MS, the modulation unit 122 can add the low-frequency signal LS into the medium-frequency signal MS in a frequency modulation (FM) manner so as to generate an interference wave signal IS (see FIG. 2C), and individually transmits the interference wave signal IS to the electrode patches 11a and 11b. Specifically, the modulation unit 122 uses the frequency modulation (FM) manner to add the low-frequency signal LS into the medium-frequency signal MS, so that the interference wave signal IS including the medium-frequency and low-frequency outputs can be obtained by using only one pair (two) of electrode patches 11a and 11b. In one embodiment, the functions of the signal generation unit 121 and the modulation unit 122 may be implemented in the form of software, hardware and/or firmware, and this disclosure is not limited thereto.

In one embodiment, the low-frequency signal LS may have a frequency between 1 Hz and 250 Hz. In one embodiment, the medium-frequency signal MS may have a frequency between 1 k Hz and 10 k Hz. In one embodiment, the medium-frequency signal MS may have a frequency between 2 k Hz and 7.8 k Hz.

The power supply unit 123 is electrically connected to the signal generation unit 121, the modulation unit 122 and the control unit 124, and the power supply unit 123 can provide an electric power for operating the electrical stimulation device 1 (including the signal generation unit 121, the modulation unit 122 and the control unit 124). In one embodiment, the power supply unit 123 may include a lithium battery. In different embodiments, the power source of the electrical stimulation device 1 may be directly from the city power system, and this disclosure is not limited thereto.

The control unit 124 is electrically connected to the signal generation unit 121, the modulation unit 122 and the power supply unit 123, and the control unit 124 is configured to control an operation of the electrical stimulation device 1. In this embodiment, the control unit 124 may include the core control components of the electrical stimulation device 1, which may at least include, for example, a central processing unit (CPU) and a memory, or any of other hardware, software or firmware.

Referring to FIG. 2A, the device body 12 of the present embodiment is provided with a mode switch button 125, and the electrical stimulation device 1 switches the operation mode thereof via the mode switch button 125. In this embodiment, the power button is also used as the mode switch button 125. In addition to the functions of power-on and power-off, this button can also be used as a switch button for operation modes. In one embodiment, the mode switch button 125 (power button) can be pressed once to turn on or off the electrical stimulation device 1, and can be pressed twice to switch the operation mode. In one embodiment, the electrical stimulation device 1 may have two or four operation modes. In one embodiment, the electrical stimulation device 1 may further include an (LED) indicator light 126 to indicate the current operation mode of the electrical stimulation device 1.

In one embodiment, the electrical stimulation device 1 has, for example, two operation modes. In the operation mode 1, the indicator light 126 displays a green light, the provided pulse frequency is 5 kHz, and the bandwidth is 200 μs. In the operation mode 2, the indicator light 126 displays a blue light, the provided pulse frequency is 2 k to 7.8 k Hz, and the bandwidth is 128 to 500 μs.

In one embodiment, the electrical stimulation device 1 has, for example, four operation modes. In the operation mode 1, the indicator light 126 displays a green light, the provided pulse frequency is 5 kHz, and the bandwidth is 200 μs. In the operation mode 2, the indicator light 126 displays a blue light, the provided pulse frequency is 2 k to 7.8 k Hz, and the bandwidth is 128 to 500 μs. In the operation mode 3, the indicator light 126 displays a flicking green light, the frequency of the carrier wave (medium-frequency wave) is 1 k Hz, the bandwidth thereof is 1 ms, and the operation frequencies include 6 Hz at the front section (bandwidth is 167 ms), 40 Hz at the middle section (bandwidth is 25 ms), and 3 Hz at the rear section (bandwidth is 333 ms). In the operation mode 4, the indicator light 126 displays a flicking blue light, the frequency of the carrier wave (medium-frequency wave) is 1 k Hz (bandwidth is 1 ms), and the operation frequency is 20 Hz (bandwidth is 50 ms). The above-mentioned embodiments with two or four operation modes are only examples, and this disclosure is not limited thereto.

In addition, the device body 12 of this embodiment is further provided with intensity adjustment buttons 127a and 127b. The intensity adjustment buttons 127a and 127b of the electrical stimulation device 1 can be used to adjust the peak-to-peak voltage (Vp-p) of the interference wave signal IS. For example, the user may press the intensity adjustment button 127a to increase the intensity, or press the intensity adjustment button 127b to decrease the intensity. In one embodiment, there are 31 levels of intensity adjustment, the starting voltage value is 0V, the intensity change of each level is, for example, 2.5V, and the maximum intensity is, for example, 77.5 Vp-p at 500 Ω.

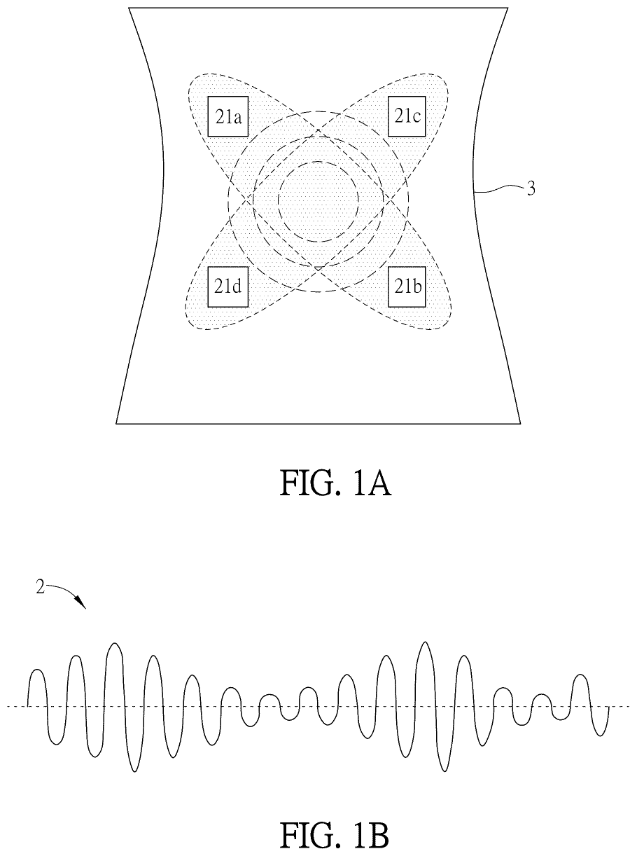

Referring to FIG. 3, in the electrical stimulation device 1 of this embodiment, the modulation unit 122 can add the low-frequency signal LS into the medium-frequency signal MS in a frequency modulation manner so as to generate the interference wave signal IS, and then transmit the interference wave signal IS to each of the electrode patches 11a and 11b. Compared with the conventional stimulator (e.g. FIG. 1A), the electrical stimulation device 1 of the present embodiment can generate a more uniform interference wave than the conventional stimulator by using only two electrode patches 11a and 11b, and the coverage area of the generated interference wave is larger than the conventional stimulator.

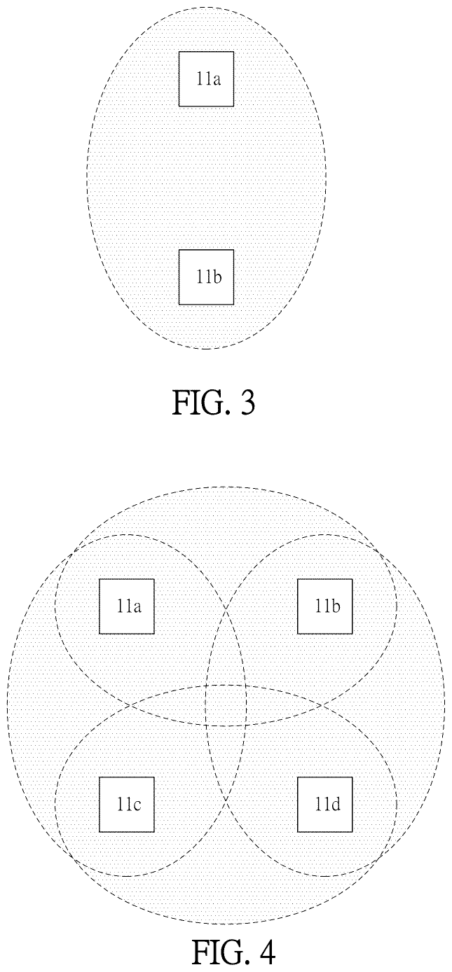

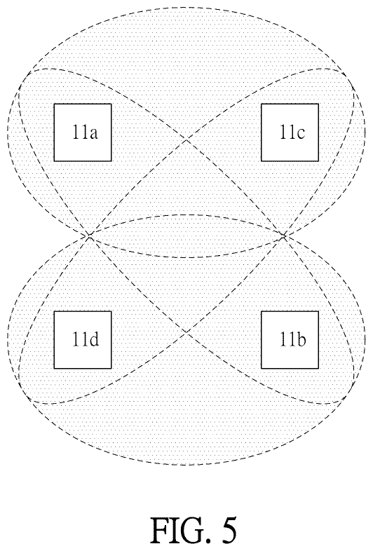

Each of the electrical stimulation devices as shown in FIG. 4 and FIG. 5 includes two pairs of electrode patches 11a to 11d (four electrode patches). The electrode patches 11a and 11b form a first pair of electrode patches, and the electrode patches 11c and 11d form a second pair of electrode patches. Referring to FIG. 4, the electrode patches 11a and 11b are located at the upper side of the drawing, and the electrode patches 11c and 11d are located at the lower side of the drawing. Referring to FIG. 5, the electrode patch 11a is located at the upper left position of the drawing, the electrode patch 11b is located at the lower right position of the drawing, the electrode patch 11c is located at the upper right position of the drawing, and the electrode patch 11d is located at the lower left position of the drawing.

As shown in FIGS. 4 and 5, compared with the conventional stimulator (e.g. FIG. 1A), the interference waves generated by the electrical stimulation device 1, which includes four electrode patches 11a to 11d, have larger coverage areas and a more uniform effect.

In summary, in the electrical stimulation device of this disclosure, the device body is electrically connected to at least two electrode patches and includes a signal generation unit and a modulation unit electrically connected to the signal generation unit. The signal generation unit outputs a low-frequency signal and a medium-frequency signal to the modulation unit, and the modulation unit adds the low-frequency signal into the medium-frequency signal in a frequency modulation manner so as to generate an interference wave signal, and individually transmits the interference wave signal to the electrode patches. Compared with the conventional electrical stimulator, the electrical stimulation device of this disclosure can use only two electrode patches to generate more uniform interference wave than the conventional electrical stimulators, and can generate the interference wave with larger coverage area.

Although the disclosure has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the disclosure.

What is claimed is:

1. An electrical stimulation device, comprising:
at least two electrode patches; and
a device body electrically connected to the electrode patches, wherein the device body comprises a signal generation unit and a modulation unit electrically connected to the signal generation unit, the signal generation unit outputs a low-frequency signal and a medium-frequency signal to the modulation unit, and the modulation unit adds the low-frequency signal into the medium-frequency signal in a frequency modulation manner so as to generate an interference wave signal, and individually transmits the interference wave signal to the electrode patches.

2. The electrical stimulation device of claim 1, further comprising:
at least two connection wires arranged corresponding to the at least two electrode patches, wherein the device body is electrically connected to the electrode patches through the at least two connection wires, respectively.

3. The electrical stimulation device of claim 1, further comprising:
a power supply unit electrically connected to the signal generation unit and the modulation unit, wherein the power supply unit provides an electric power for operating the electrical stimulation device.

4. The electrical stimulation device of claim 3, further comprising:
a control unit electrically connected to the signal generation unit, the modulation unit and the power supply unit, wherein the control unit is configured to control an operation of the electrical stimulation device.

5. The electrical stimulation device of claim 1, wherein the low-frequency signal has a frequency between 1 Hz and 250 Hz.

6. The electrical stimulation device of claim 1, wherein the medium-frequency signal has a frequency between 1 k Hz and 10 k Hz.

7. The electrical stimulation device of claim 1, wherein the device body is configured with a mode switch button for switching an operation mode of the electrical stimulation device.

8. The electrical stimulation device of claim 1, wherein the device body is configured with an intensity adjustment button for adjusting a peak-to-peak voltage value of the interference wave signal of the electrical stimulation device.

9. The electrical stimulation device of claim 1, wherein the electrical stimulation device comprises four of the electrode patches.

10. The electrical stimulation device of claim 1, wherein the electrical stimulation device is a transcutaneous electrical nerve stimulator (TENS).
